(19) United States
(12) Patent Application Publication
(10) Pub. No.: US 2012/0158025 A1
(43) Pub. Date: Jun. 21, 2012

(54) ANASTOMOSIS DEVICE AND RELATED METHODS

(76) Inventors: Christopher Anderson, New Hope, MN (US); Patricia M. Derus, Rogers, MN (US)

(21) Appl. No.: 13/326,936

(22) Filed: Dec. 15, 2011

Related U.S. Application Data
(60) Provisional application No. 61/423,487, filed on Dec. 15, 2010.
(60) Provisional application No. 61/423,746, filed on Dec. 16, 2010.

(51) Int. Cl. A61B 17/11

(52) U.S. Cl. 606/153

(57) ABSTRACT

An anastomosis device that includes a distal end portion is provided. The distal end portion of the catheter can include a stent-like drainage feature to prevent the formation of blood clots and to improve the flow of urine out of the catheter. The device can further include a balloon device having a thickened wall region to prevent puncturing or damage from the catheter tines.

### PRIORITY CLAIM AND RELATED APPLICATIONS

The present application claims the benefit of U.S. Provisional Application No. 61/423,487 filed Dec. 15, 2010, and entitled “ANASTOMOSIS DEVICE AND RELATED METHODS” and U.S. Provisional Application No. 61/423,746 filed Dec. 16, 2010 and entitled “ANASTOMOSIS DEVICE AND RELATED METHODS”, both of which are incorporated herein in their entirety by reference.

### FIELD OF THE INVENTION

The present application generally relates to anastomosis devices for use in reconnecting urological lumens. More specifically, the present application is directed to anastomosis devices and related methods of avoiding clotting and plugging of a drainage lumen during use of the anastomosis device in reconnecting severed urological lumens.

### BACKGROUND OF THE INVENTION

There exist a variety of urological procedures where a step of the procedure involves severing a biological lumen and/or removing tissue. One such urological procedure is a radical prostatectomy, wherein a surgeon removes all or most of a patient's prostate. Because the urethra travels through the prostate immediately before reaching the bladder, an upper part of the urethra is also removed in the surgery. Once the prostate is removed, the radical prostatectomy procedure leaves a severed urethral stump and a severed bladder neck. To restore proper urinary functions, the bladder and the urethra must be reconnected through connection of the severed urethral stump to the severed bladder neck.

On conventional method by which this reconnection is accomplished is through the use of sutures wherein a surgeon executes delicate suturing operations with tiny, fine needles. The use of sutures as part of the reconnection procedure can be a difficult and often technique-sensitive task. Many factors can make this task difficult, including the typically small amounts of tissue to work with during reconnection (at the urethral stump and at the bladder neck), proximal ureters at the bladder, and a proximal nerve bundle and sphincter at the urethral stump. All of these add up to a complicated and delicate suturing procedure that, if not performed properly, can result in complications such as leakage, difficulty in healing or failure to heal, incontinence, or even impotence. Specific problems include necrosis of the sutured tissues; stricture of the urethra so as to impedes the flow of fluid through it; and a urethra-bladder connection that is not fluid-tight. In addition, methods of suturing the urethra to the bladder allow for accidental or inadvertent piercing of the nearby neurovascular bundle, which can cause incontinence or impotence.

The use of an anastomosis device in a radical prostatectomy procedure allows a physician to complete the reconnection procedure without the need, and consequently, the potential problems associated with suturing.

### SUMMARY OF THE INVENTION

The present application is directed to an anastomosis device that includes a catheter body and tissue approximating structure. The tissue approximating structure can be used to cause or maintain contact between severed portions of tissue to allow or cause the severed tissue surfaces to heal together, instead of using sutures. The device may be used, for example, in performing procedures such as a vesico-urethral anastomosis in association with a radical prostatectomy, or with an end-to-end urethral anastomosis. Advantageously, the device, including the tissue approximating structure, can be left installed during the healing process to function to allow the tissue to heal while at the same time functioning to drain urine from the bladder.

Embodiments of anastomosis devices of the invention, in addition to the tissue approximating structure, can include features and structures that function to drain the bladder, e.g., through a drain lumen, while at the same time functioning as a tissue approximating structure that causes contact between severed tissue to allow the severed tissues to heal together. Methods of the invention can use such an anastomosis device, including both a draining function and a tissue approximating function, to accomplish healing of the anastomosis, without sutures, and draining of the bladder, with the single anastomosis device and preferably without removing or replacing the device during or after the procedure until healing is complete. The anastomosis device can be installed during or after a radical prostate removal procedure, and can remain installed with the bladder-draining function and the tissue-approximating function in effect until the anastomosis is completely healed and the severed tissue, e.g., bladder and urethra, are re-connected. Thus, an advantage associated with inventive methods and devices can be that the anastomosis device performs dual functions when installed during and following an anastomosis procedure, of draining the bladder and functioning as a tissue approximating structure, at the same time.

Also advantageously, inventive methods and devices avoid the need to use sutures to connect severed tissue such as a bladder neck and urethral stump. The ability to avoid sutures provides very significant advantages of avoiding the potential for damage to surrounding tissues and nerves that can be caused by installation of sutures using a needle. Such damage can include, for example, damage to ureters at the bladder or damage to the sphincter or nerves located in the perineal floor. Damage to any of these tissues has the potential to cause incontinence or impotency. Additionally, installing sutures is a difficult and technique-sensitive process that must be performed in a confined space and that would be avoided if possible based on other alternatives. Thus, the invention offers the very significant advantage of eliminating the need to use sutures to re-attach severed tissues, and the attendant potential damage to those sensitive proximal tissues and nerves and the possibility of incontinence or impotency.

The terms “tissue approximating” and simply “approximating” refer to a process of bringing or holding body tissues in contact for healing. Examples include: the process of bringing severed surfaces of a bladder neck and a urethral stump, or two opposing severed urethral tissues, into contact for healing; and the process of holding severed surfaces of a bladder neck and a urethral stump, or two opposing severed urethral tissues, together for healing.

An aspect of the device relates to an anastomosis device comprising a hollow, elongate, flexible catheter body having a proximal end and a distal end; an inflatable balloon at the distal end; a drainage lumen connected to the distal end; and tissue approximating structure on the catheter body on a proximal side of the balloon at a location to contact severed tissue during an anastomosis procedure.

Another aspect of the invention relates to a method of performing urethral anastomosis. The method comprises inserting a portion of an anastomosis device into the urethra, the anastomosis device comprising tissue approximating structure and a distal end comprising a balloon; inflating the balloon in the bladder; and using the tissue approximating structure to hold severed tissue together. Preferably, the device further comprises drainage apertures for draining a bladder and the method comprises draining a bladder.

Certain embodiments of the invention can include tissue approximating structure having one or more tines extending therefrom. The tines can be formed as sharp elongate, tubular straight or curved, or fine metal barbs that movably extend from the distal end of the catheter body at a location to facilitate contact and penetration of the urethral stump proximal tissue in the adjacent perineal floor, urethra, or bulbar urethra.

Still other embodiments can include one or more catheters configured to prevent the formation of blood clots and, thereby, improving the flow of urine out of the catheter. The catheter can include a stent-like or like drainage feature provided proximate the distal tip and catheter balloon. The drainage feature can be constructed similar to Urolume™, with a silicone insert molded at both ends of the feature for a soft tip to facilitate insertion and bonding to other components.

The drainage feature can be inserted in a compressed state and then actuated or elongated to lengthen the feature. This will take the tip from a solid tube state to a Nitinol wire state with expanded holes or gaps for drainage. Alternatively, the drainage feature can be inserted in the elongated state. By pulling or otherwise activating the feature (e.g., with expansion of bladder tines), the feature is pulled inward or shorter such that it generally bubbles or expands out in the middle. This will give the feature a bulged profile to provide expanded gaps to make it more difficult for blood clots to fully cover and plug the tip.

In various embodiments, the expandable balloon proximate or at the distal portion of the device can be molded with a base portion that is measurably thicker than the other portions of the balloon. This will assist in preventing puncturing or other breaches of the region of the balloon coming into contact with sharp devices, such as the device tines. The shape of the balloon can be defined to accommodate the thicker base.

Various embodiments of the catheter can include a removable or replaceable lumen to provide reestablishment and maintenance of the urine flow through the device in the event the original or previously provided catheter lumen becomes blocked or obstructed. A removable and replaceable lumen allows for correction of any blockage without disturbing the anastomotic site.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE DRAWINGS

The invention can be completely understood in consideration of the following detailed description of various embodiments of the invention in connection with the accompanying drawings, in which:

FIG. 1 is a perspective view of an anastomosis device of the prior art.

FIG. 2 is a schematic view to illustrate general aspects of radical prostate removal.

FIG. 3 is a schematic view of a portion of an anastomosis device in accordance with embodiments of the present invention.

FIG. 4 is a schematic view of a distal portion of an anastomosis device, showing a distal stent-like device in a contracted state, in accordance with embodiments of the present invention.

FIG. 5 is a schematic view of a distal portion of an anastomosis device, showing a distal stent-like device in an expanded state, in accordance with embodiments of the present invention.

FIG. 6 is a schematic view of a distal portion of an anastomosis device, showing a distal stent-like device in an extended state, in accordance with embodiments of the present invention.

FIG. 7 is a schematic view of a distal portion of an anastomosis device, showing a distal stent-like device in a compressed state, in accordance with embodiments of the present invention.

FIG. 8 is a schematic view of a distal portion of an anastomosis device, showing a distal balloon device having a thickened bottom wall portion, in accordance with embodiments of the present invention.

FIG. 9 is a schematic view of a catheter device and removable drainage lumen in accordance with embodiments of the present invention.

FIG. 10 is a schematic view of a removable drainage lumen being inserted into a catheter device in accordance with embodiments of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF PREFERRED EMBODIMENTS

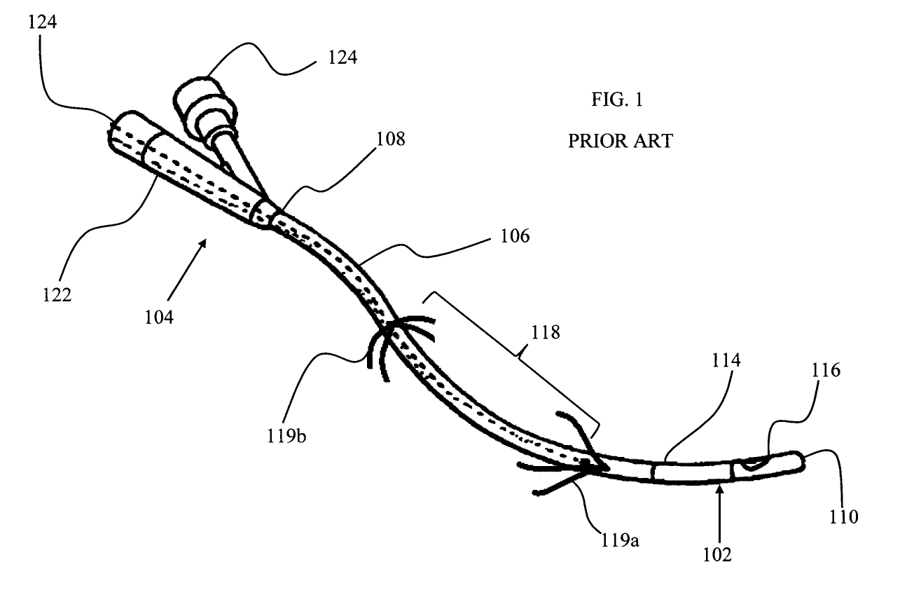

Referring generally to FIGS. 1-7, an anastomosis device useful according to the invention can be any anastomosis device adapted to perform one or more of the methods described herein, e.g., an anastomosis method associated with a radical prostatectomy. An example of a useful design, generally, is the type sometimes referred to as a Foley catheter that has been constructed to include modified features as also presented in the present description including tissue approximating structure, ports, fluid conduits, and the like.

An anastomosis device of the invention can include a hollow, elongate, flexible catheter body having a proximal end and a distal end. An inflatable balloon can be located near the distal end, and an inflation lumen for inflating the balloon can extend to the balloon along the catheter body, e.g., along a portion or all of the catheter body from the proximal end to the balloon. During use, the balloon can rest against the neck of the bladder to prevent urine from entering the neck and to prevent urine from contacting the anastomosis site. Urine at the anastomosis site has the potential to cause difficulties in healing or to cause a stricture, among other deleterious effects. With the balloon blocking the bladder neck during use, urine will pool in the bladder and can be drained from the bladder, for example, using one or more draining apertures at the distal end of the anastomosis device connected to a drainage lumen. A drainage lumen can extend from one or more drainage apertures near the distal end, e.g., from apertures at the distal end to a location that is at or near the proximal end.

Overall, an anastomosis device can contain various lumens (e.g., for inflating a balloon, drainage, providing a fluid channel or conduit to the tines, for containing actuating mechanisms for tissue approximating structure, etc.) and actuating mechanisms running along at least a partial length of the catheter body. The mechanisms and lumens can be arranged in any useful adaptation, such as coaxially, side-by-side, or according to any other useful configuration. A lumen or a mechanism (e.g., actuating mechanism) that runs along at least a portion of the length of the catheter body may be diverted at the proximal end of the catheter body to a port that provides access to the lumen or mechanism during use. Further, the various devices, components, mechanisms, and methods disclosed in U.S. Patent Publication No. 2007/0219584 can be employed, alone or in combination, with the present invention and, therefore, the above-identified publication is incorporated herein by reference in its entirety.

Referring generally to FIGS. 1, 2 and 3, an anastomosis device 100 of the prior art can comprise a catheter portion 102 and an actuation portion 104. Catheter portion 102 generally comprises a catheter body 106 having a proximal connection portion 108 and a distal end portion 110. Catheter body 102 is generally constructed of a soft plastic or rubber tube. Located proximate to distal end portion 110, the catheter body can include an inflation balloon 114, a drainage aperture 116 and a tissue approximating structure 118. Tissue approximating structure 118 can comprise a single structure or multiple structures generally positioned along the catheter body 106 between the distal end portion 110 and the proximal connection portion 108. Tissue approximating structure 118 can include multiple sharp elongate tines that can extend from and retract into catheter body 106 at a location that allows contact and optional penetration of adjacent tissue structures. Tissue approximation structure 118 can comprise a distalmost approximating structure 119a and a proximalmost approximating structure 119b.

Referring again to FIG. 1, proximal connection portion 108 of the anastomosis device 100 can operably connect to actuation portion 104 at an actuation body 122. Actuation body 122 generally possesses an increased diameter than catheter body 106. Actuation body 122 can include one or more ports 124 that extend or otherwise protrude from the actuation body 122 and generally provide access to distinct lumens defined within the catheter body 106 that can provide for inflation of the inflation balloon 114, drainage from the drainage aperture 116 or for control guidewires to operably connect to the tissue approximating structures 118.

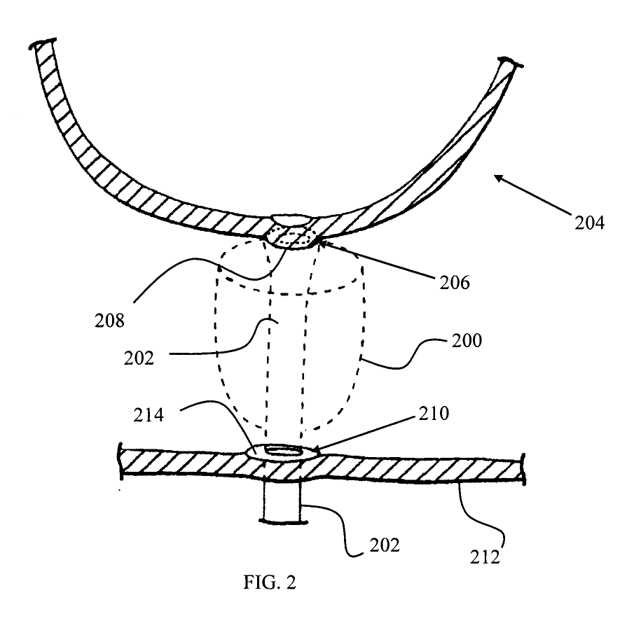

According to embodiments of the invention, the tissue approximating structure 118 of anastomosis device 100 can be used to place or hold a cut or severed tissue or tissue surface in place for healing, as well as to inject or provide drugs or cell mixtures into target tissue to facilitate healing and overall tissue health. Referring generally to FIG. 2, a radical prostatectomy procedure includes removal of the prostate 200 (indicated in dashes) and urethra 202, leaving bladder 204 with bladder neck 206 having a severed tissue surface 208 at one end of removed prostate 200, and a urethral stump 210 extending from perineal floor 212, with urethral stump 210 having severed tissue surface 214 opposing a severed tissue surface 208 of bladder neck 206. While the following description presents inventive devices and methods primarily in the context of vesico-urethral anastomosis relating to radical prostatectomy, it will be apparent that the invention can be applied to a variety of other procedures that benefit from tissue approximating structures and in particular where a fluid flow is also desired, such as drainage of urine. A specific example is an end-to-end urethral anastomosis procedure.

The tissue approximating structure 118 of the anastomosis device 100 can be a structure of the device that can be used to cause contact between severed tissues, such as severed urethral tissues, or such as severed tissue of the bladder or bladder neck with severed tissue of the urethral stump or perineal floor, or alternatively or additionally to hold severed tissue surfaces in contact with each other for healing. In addition to the tissue approximating structure 118 including the distalmost approximating structure 119a and the proximalmost approximating structure 119b, the tissue approximating structure 118 can include, for example, one or multiple inflation balloons 114 or balloon-like structures that can be placed against the inside of the bladder or underneath the perineal floor to bring the severed bladder neck tissue into contact with the severed tissue surface of the urethral stump. The distalmost approximating structure 119a and the proximalmost approximating structure 119b can include elongate structures such as needles or tines (solid or hollow), prods, probes, or the like, which may have a blunt or a sharp end and may selectively extend or protrude from an aperture in the catheter body 106 at a location where the structure can function as an approximating structure, e.g., at the distal end portion 110 of the anastomosis device 100 where the distalmost approximating structure 119a and the proximalmost approximating structure 119b structure will be near the bladder or perineal wall (when installed), or at a severed urethra below the perineal floor (when installed). The tissue approximating structure 118 does not require and can specifically exclude and avoid the use of conventional sutures.

An example of a useful tissue approximating structure 118 can be in the form of a sharp or blunt elongate structure (e.g., a sharp-ended needle or tine) that can be movably extended from an aperture proximate the distal end portion 110 of, or along a length of, the catheter body 106, to thereby contact and optionally penetrate into or through one or more of a tissue of the bladder, bladder neck, urethra, bulbar urethra, urethral stump, or perineal floor. These structures can serve to place opposing severed tissue surface into contact for healing, and preferably also to hold the tissues in contact with each other during the healing period. Certain embodiments of the invention can include sharp or blunt elongate tissue approximating structure 118 (e.g., a sharp-ended needle or tine) that can be movably extended from an aperture proximate the distal end portion 110 of the catheter body 106 to place opposing severed tissue of the bladder neck into contact with a severed tissue surface of the urethral stump, or vice versa, and preferably also to hold the tissues in contact with each other during the healing period.

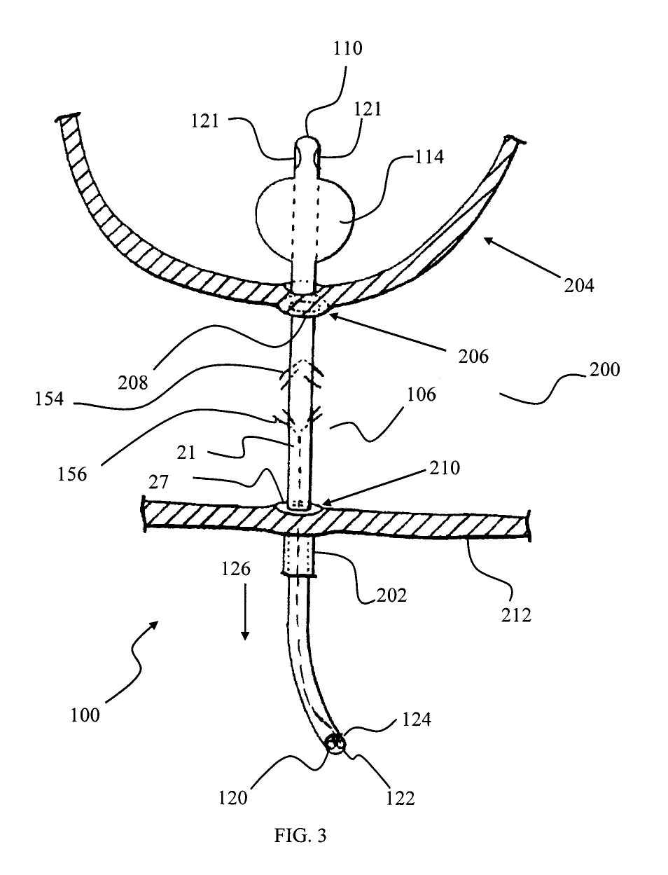

Referring to FIG. 3, prostate 200 has been removed to leave the severed tissue surface 214 of the urethral stump 210 opposing the severed tissue surface 208 of bladder neck 206. Anastomosis device 100 is slidably advanced installed through urethral stump 210 and bladder neck 206. As shown, the anastomosis device 100 can also include drain lumen 120, one or more actuation lumens 122, and drain apertures located between the distal end portion 110 of the catheter body 106 and inflation balloon 114. Inflation balloon 114 is inflated, after insertion into the bladder 204, by a flow of fluid through an inflation lumen 124. Pressure (e.g., traction as shown by arrow 126) can then be applied through the length of anastomosis device 100 to produce a pressure against the inside of bladder 204 using the now inflated, inflation balloon 114.

In related embodiments of devices according to FIGS. 1-3, the distalmost approximating structure 119a and the proximalmost approximating structure 119b can take the form of distal tines 154 or proximal times 156 respectively, or alternatively, injection needles. The distal and proximal tines 154, 156 can be mounted within the actuation lumen 122 such that one or more a guidewires 158 (shown in dashes) allows a medical professional to selectively deploy or retract the distal and proximal tines 154, 156 at the actuation portion 104. Various other fluid channeling and configurations are envisioned for use with the present invention. It is noted that various other catheter, conduit, port and tine configurations can be employed to facilitate introduction of drugs or cells into target tissue via needles or tines to provide local or site-specific delivery.

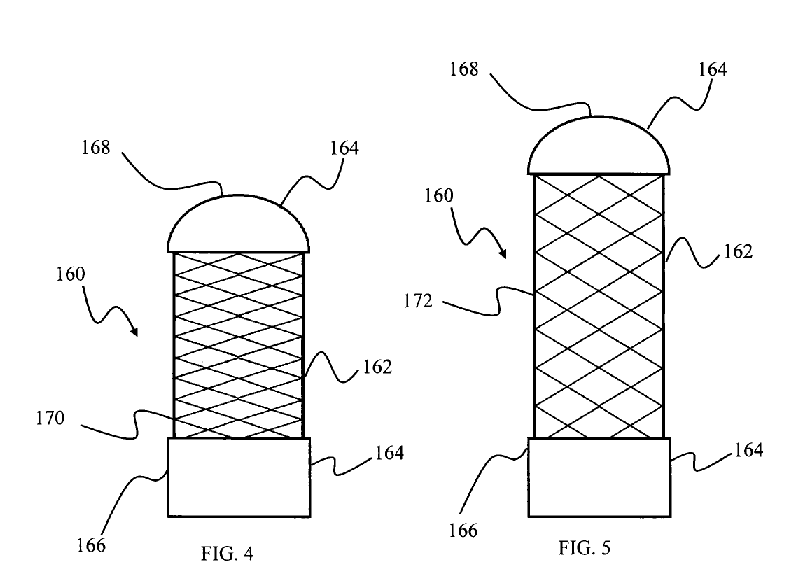

In various embodiments, as shown in FIGS. 4-7, the distal end portion 110 of catheter body 106 can include a stent or stent-like drainage feature 160 configured to prevent the formation of blood clots and, thereby, improving the flow of urine out of the catheter. The drainage feature 160 can be provided proximate the distal tip portion 110 and inflation balloon 114. The drainage feature 160 can be constructed similar to Urolume™, with a stent-like drainage body 162 and a silicone insert 164 molded at an attachment tip 166 and a guide tip 168 located at opposed ends of the drainage body 162. Guide tip 168 provides a soft tip to facilitate insertion while the attachment tip allows for bonding of the drainage body 162 to the distal end portion 110 of the catheter body 106.

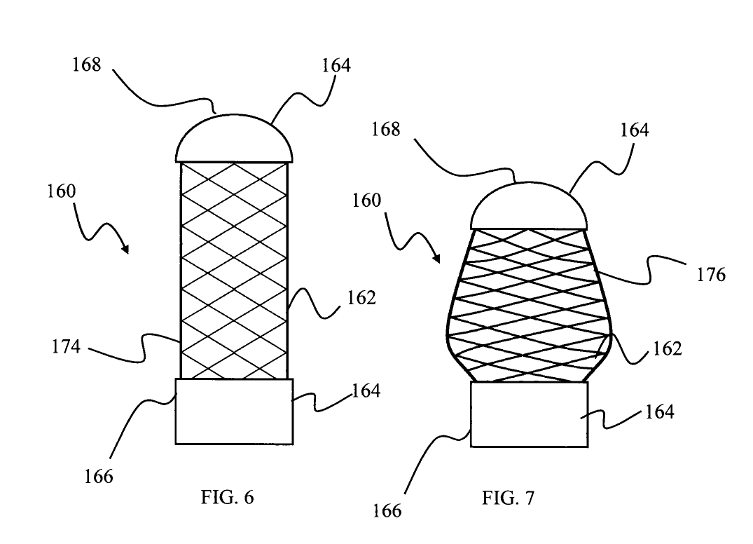

The drainage feature 160 can be inserted in a compressed state 170 shown in FIG. 4 and then actuated or elongated to lengthen to assume an expanded state 172 as shown in FIG. 5. The actuation/elongation takes the drainage body 162 from a first solid orientation to a second expanded orientation, i.e., from a solid tube state to a Nitinol wire state with expanded holes or gaps for drainage. Alternatively, the drainage feature 160 can be inserted in an elongated state 174 as shown in FIG. 6 wherein pulling or otherwise activating the drainage feature 60 (e.g., with actuation of the distal and proximal tines 154, 156), the drainage feature 160 is pulled inward or shorter such that it generally bubbles or expands out in the middle to assume a bulged state 176 as shown in FIG. 7. With the drainage body 162 expanded to the bulged state 176, the likelihood of blood clots fully covering and blocking the drainage feature 160 can be reduced.

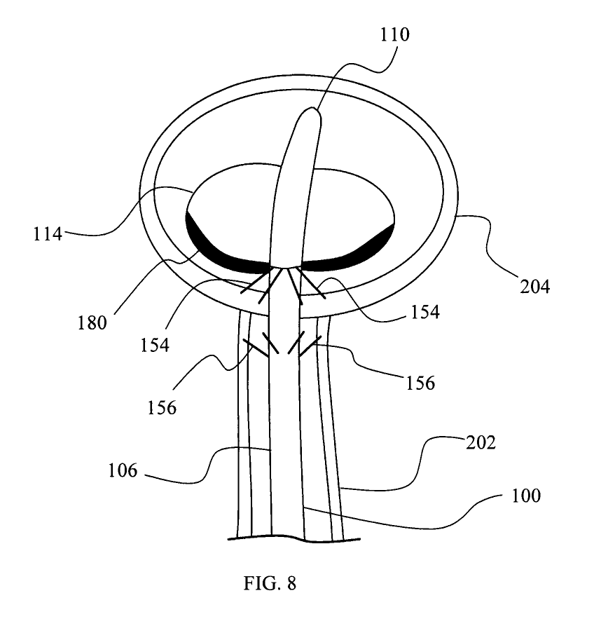

In various embodiments, as depicted in FIG. 8, the inflation balloon 114 located either proximate or at the distal end portion 110 of the anastomosis device 100 can be molded with a base portion 180 that is measurably thicker than the other portions of the balloon. This will assist in preventing puncturing or other breaches of the region of the balloon coming into contact with sharp devices, such as, for example, the distal and proximal tines 154, 156. The shape of the inflation balloon 114 can be defined to accommodate the thicker base portion 180. This protective thickening of the base 180 can also allow for employment of additional distal and proximal tines 154, 156 (e.g., going from 6 each to 9 each or more) proximate the base portion 180 of inflation balloon 114 to promote fixation, push back and fluid flow potential.

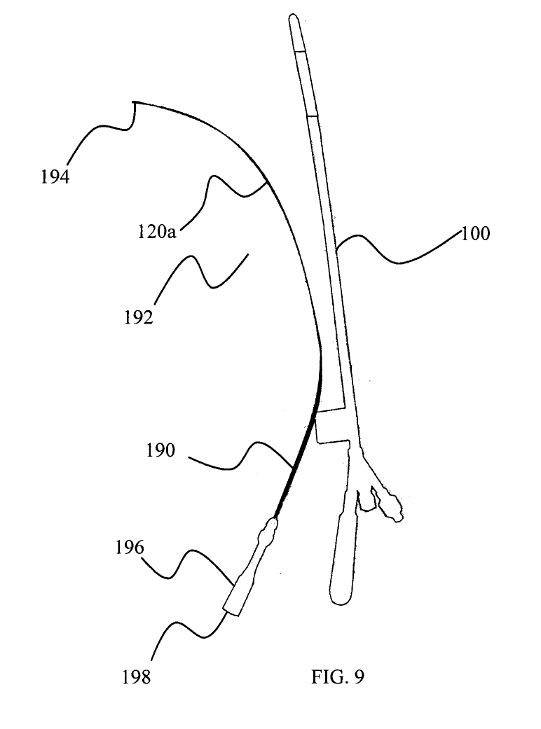

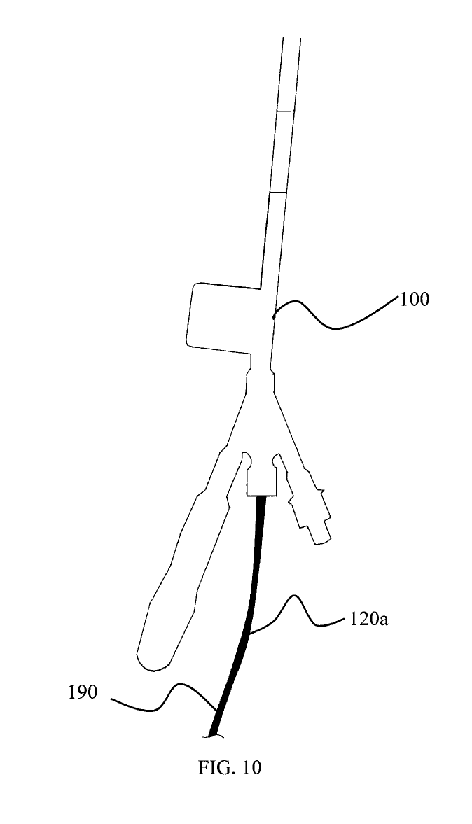

As shown in FIGS. 9 and 10, the drain lumen 120 can comprise a removable or replaceable drain lumen 120a provided through the catheter body 106. The removable or replaceable lumen 120a can provide reestablishment and maintenance of urine flow through the anastomosis device 100 in the event the original or previously provided catheter lumen 120 becomes blocked or obstructed. A removable and replaceable lumen 120a allows for correction of any blockage without disturbing the anastomotic site. The removable lumen 120a can be appropriately sized and shaped, and can include a tube 190 having a tube length 192 defined between a distal insertion portion 194 and a proximal manipulation portion 196. Distal insertion portion 194 can be inserted along all or part of the length of the catheter body 106 such that lumen 120a extends to a desired point within drain lumen 120. Proximal manipulation portion 196 provides a medical professional with the capability to insert, manipulate and adjust the placement of tube 190 within drain lumen 120. Proximal manipulation portion 196 can provide additional input ports 198 so as to provide access to drain lumen 120 and to supplement the input ports provided as part of actuation portion 104 of the anastomosis device 100.

The catheter and its componentry may be made of materials normally used and known to be useful for such devices, including relatively inert and biocompatible materials. For example, a catheter body may be prepared from a flexible plastic or polymeric material. Examples of materials that may be useful for a catheter body can include silicones, latex, rubbers, polyurethanes, and combinations of these or other materials. A tissue approximating structure can be made from these or other materials, including relatively rigid plastics, polymers, or metals, optionally including bioresorbable materials such as bioresorbable polymers. Examples of metals include stainless steel, nitinol, titanium, tantalum, as well as alloys or combinations of these materials.

In general, a catheter can be used during urethral anastomosis procedures such as that associated with a radical prostatectomy, e.g., vesico-urethral anastomosis, with the catheter functioning to remove urine from the bladder after the procedure. The anastomosis device can be used by inserting the elongate flexible catheter body through the urethra and into the bladder. A portion of the distal end of the device becomes located inside of the bladder where the balloon can be inflated and where the drainage lumen can be used to drain the bladder and keep urine out of the bladder during and subsequent to the procedure. The bladder can preferably be drained of urine during the procedure and during the healing period following the procedure, because urine is preferably kept away from the site of anastomosis to facilitate healing, and also to prevent urine from creating pressure within the bladder.

Certain embodiments can include various drugs and/or cellular mixtures, e.g., cells and adipose tissue, coated onto, or impregnated or otherwise provided with structures of the device. Again, these structures can come into contact with target tissue to increase the speed and degree of healing, and to promote overall tissue health. For instance, the tines or distal tip, with or without fluid injection capabilities, can include the cellular mixtures. In other embodiments, foam wrappings, protrusions, or surface portions of the device can include the drugs and/or cellular mixtures.

Generally, a method of the invention can include a step of performing a radical prostatectomy, such as by a retropubic technique, a laparoscopic technique, or a transperineal technique. These techniques leave a bladder neck and a urethral stump for re-attachment. The distal portion of the anastomosis device may optionally be partially installed during the prostatectomy procedure, e.g., up to the perineal floor, or may be installed to that point afterward. Following removal of the prostate, the catheter body of the distal end of the device is passed through the urethral stump and then through the bladder neck. From there, the technique can include inflating the balloon inside of the bladder, using tissue approximating structure (including tines 54, 46) to place the severed tissue surfaces of the urethral stump and the bladder neck into contact, and injecting drugs or cells (such as a cell mixture) into the target tissue through the engaged injection tines.

All patents, patent applications, and publications cited herein are hereby incorporated by reference in their entirety as if individually incorporated, and include those references incorporated within the identified patents, patent applications and publications.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative embodiments.

1. An anastomosis device, comprising:
a catheter body including a distal treatment end and a proximal biasing end, the catheter body including a drainage lumen fluidly extending between the distal treatment end and the proximal biasing end; and
a drainage feature mounted at the distal treatment end, the drainage feature including a drainage body having an attachment tip for bonding the drainage body to the distal treatment end and a guide tip, the drainage body adapted so as to assume a first solid orientation during insertion of the distal treatment end to a desired treatment location and to selectively assume a second expanded orientation during a period of tissue retention.

2. The anastomosis device of claim 1, wherein the attachment tip and the guide tip comprise a molded silicone.

3. The anastomosis device of claim 1, wherein the second expanded orientation comprises an elongated orientation.

4. The anastomosis device of claim 1, wherein the second expanded orientation comprises an outwardly bulged orientation.

5. The anastomosis device of claim 1, wherein the catheter body further comprises a tissue approximation structure including a deployment member and a plurality of tines, said plurality of tines being outwardly deployable when actuation by the deployment member.

6. The anastomosis device of claim 5, wherein the deployment member is operably coupled to the drainage body such that the actuation member selectively causes the drainage body to transition between the first solid orientation and the second expanded orientation.

7. The anastomosis device of claim 6, wherein the tissue approximation structure further comprises an inflation balloon, said inflation balloon being fluidly attached to an inflation lumen within the catheter body.

8. The anastomosis device of claim 7, wherein the inflation balloon is mounted on the catheter body between the plurality of tines and the distal treatment end, the inflation balloon including a proximal base portion facing the plurality of tines, wherein the proximal base portion has a wall thickness greater than a distalmost portion of the inflation balloon.

9. The anastomosis device of claim 1, further comprising:
a removable drain tube, the removable drain tube having a tube length defined between a distal insertion portion and a proximal manipulation portion wherein the distal insertion portion is slidably insertable into the drain lumen.

10. A method for limiting effects of clotting during an anastomosis procedure, comprising:
providing an anastomosis device including a catheter body having a distal treatment end, a proximal biasing end and a drainage lumen fluidly extending between the distal treatment end and the proximal biasing end;
attaching a drainage body to the distal treatment end;
advancing the distal treatment end to a desired treatment location, wherein the drainage body is arranged in a first solid orientation during insertion; and
adjusting the drainage body to assume a second expanded orientation during a period of tissue retention.

11. The method of claim 10, wherein adjusting the drainage body to assume the second expanded orientation, comprises:
adjusting the drainage body to assume an elongated orientation.

12. The method of claim 10, wherein adjusting the drainage body to assume the second expanded orientation, comprises:
adjusting the drainage body to assume an outwardly bulged orientation.

13. The method of claim 10, wherein attaching the drainage body to the distal treatment end, comprises:
molding an attachment tip of the drainage body to the distal treatment end.

14. The method of claim 10, further comprising:
actuating an approximation structure on the catheter body to capture and retain tissue during the period of tissue retention.

15. The method of claim 14, wherein the approximation structure comprises a plurality of tines retained within the catheter body, the plurality of tines operably coupled to a deployment member, wherein the step of actuating the approximation member, further comprises:
biasing the deployment member such that the plurality of tines are directed outwardly from the catheter body.

16. The method of claim 15, wherein biasing the deployment member causes the drainage body to adjust from the first solid orientation to the second expanded orientation.

17. The method of claim 10, further comprising:
inserting a removable drain tube into the drain lumen;
removing the removable drain tube during period of tissue retention when flow is disrupted through the removable drain tube; and
inserting a second removable drain tube into the drain lumen.
